(19) United States
(12) Patent Application Publication
(10) Pub. No.: US 2019/0262405 A1
(43) Pub. Date: Aug. 29, 2019

(54) PLURIPOTENT STEM CELL-DERIVED OLIGODENDROCYTE PROGENITOR CELLS FOR THE TREATMENT OF SPINAL CORD INJURY

(71) Applicant: Asterias Biotherapeutics, Inc., Fremont, CA (US)

(72) Inventors: Edward D. Wirth, III, Mountain View, CA (US); Jane S. Lebkowski, Portola Valley, CA (US)

(73) Assignee: Asterias Biotherapeutics, Inc., Fremont, CA (US)

(21) Appl. No.: 16/333,566

(22) PCT Filed: Sep. 14, 2017
(86) PCT No.: PCT/US17/51677

§ 371 Date: Mar. 14, 2019

Related U.S. Application Data
(60) Provisional application No. 62/518,591, filed on Jun. 12, 2017.
(60) Provisional application No. 62/449,580, filed on Jan. 23, 2017.
(60) Provisional application No. 62/394,226, filed on Sep. 14, 2016.

(51) Int. Cl. A61K 35/30; C12N 5/079; A61K 9/00; A61P 25/00; A61P 37/06; C12N 5/0735

(52) U.S. Cl. CPC A61K 35/30; C12N 5/0622; C12N 5/0606; A61P 25/00; A61P 37/06; A61K 9/0019

(57) ABSTRACT

Methods and compositions for making and using pluripotent stem cell-derived oligodendrocyte progenitor cells for the treatment of spinal cord injury are disclosed.

### CROSS-REFERENCE TO RELATED APPLICATIONS

This application claims priority to U.S. Provisional Application No. 62/394,226, filed Sep. 14, 2016; U.S. Provisional Application No. 62/449,580, filed Jan. 23, 2017; and U.S. Provisional Application No. 62/518,591, filed Jun. 12, 2017, the contents of which are hereby incorporated by reference in their entireties.

### FIELD

The present disclosure relates to the field of stem cell biology and oligodendrocyte progenitor cells. More specifically, the present disclosure relates to oligodendrocyte progenitor cell compositions and methods of using the same.

### BACKGROUND

Over 12,000 Americans suffer a spinal cord injury (SCI) each year, and approximately 1.3 million people in the United States are estimated to be living with a spinal cord injury. Traumatic SCI most commonly impacts individuals in their 20s and 30s, resulting in a high-level of permanent disability in young and previously healthy individuals. Individuals with SCI not only have impaired limb function, but suffer from impaired bowel and bladder function, reduced sensation, spasticity, autonomic dysreflexia, thromboses, sexual dysfunction, increased infections, decubitus ulcers and chronic pain, which can each significantly impact quality of life, and can even be life threatening in some instances. The life expectancy of an individual suffering a cervical spinal cord injury at age 20 is 20-25 years lower than that of a similarly aged individual with no SCI (NSCISC Spinal Cord Injury Facts and Figures 2013).

The clinical effects of spinal cord injury vary with the site and extent of damage. The neural systems that may be permanently disrupted below the level of the injury not only involve loss of control of limb muscles and the protective roles of temperature and pain sensation, but impact the cardiovascular system, breathing, sweating, bowel control, bladder control, and sexual function (Anderson K D, Fridén J, Lieber R L. Acceptable benefits and risks associated with surgically improving arm function in individuals living with cervical spinal cord injury. Spinal Cord. 2009 April; 47(4):334-8.) These losses lead to a succession of secondary problems, such as pressure sores and urinary tract infections that, until modern medicine, were rapidly fatal. Spinal cord injury often removes those unconscious control mechanisms that maintain the appropriate level of excitability in neural circuitry of the spinal cord. As a result, spinal motoneurons can become spontaneously hyperactive, producing debilitating stiffness and uncontrolled muscle spasms or spasticity. This hyperactivity can also cause sensory systems to produce chronic neurogenic pain and paresthesias, unpleasant sensations including numbness, tingling, aches, and burning. In recent polls of spinal cord injury patients, recovery of ambulatory function was not the highest ranked function that these patients desired to regain, but in many cases, relief from the spontaneous hyperactivity sequelae was paramount (Anderson K D, Fridén J, Lieber R L. Acceptable benefits and risks associated with surgically improving arm function in individuals living with cervical spinal cord injury. Spinal Cord. 2009 April; 47(4):334-38).

There are multiple pathologies observed in the injured spinal cord due to the injury itself and subsequent secondary effects due to edema, hemorrhage and inflammation (Kakulas B A. The applied neuropathology of human spinal cord injury. Spinal Cord. 1999 February; 37(2):79-88). These pathologies include the severing of axons, demyelination, parenchymal cavitation and the production of ectopic tissue such as fibrous scar tissue, gliosis, and dystrophic calcification (Anderson D K, Hall E D. Pathophysiology of spinal cord trauma. Ann Emerg Med. 1993 June; 22(6):987-92; Norenberg M D, Smith J, Marcillo A. The pathology of human spinal cord injury: defining the problems. J. Neurotrauma. 2004 April; 21(4):429-40). Oligodendrocytes, which provide both neurotrophic factor and myelination support for axons are susceptible to cell death following SCI and therefore are an important therapeutic target (Almad A, Sahinkaya F R, Mctigue D M. Oligodendrocyte fate after spinal cord injury. Neurotherapics 2011 8(2): 262-73). Replacement of the oligodendrocyte population could both support the remaining and damaged axons and also remyelinate axons to promote electrical conduction (Cao Q, He Q, Wang Yet et al. Transplantation of ciliary neurotrophic factor-expressing adult oligodendrocyte precursor cells promotes remyelination and functional recovery after spinal cord injury. J. Neurosci. 2010 30(8): 2989-3001).

AST-OPC1 is a population of oligodendrocyte progenitor cells (OPCs) that are produced from human embryonic stem cells (hESCs) using a specific differentiation protocol (Nistor G I, Totoiu M O, Haque N, Carpenter M K, Keirstead H S. Human embryonic stem cells differentiate into oligodendrocytes in high purity and myelinate after spinal cord transplantation. Glia. 2005 February; 49(3):385-96). AST-OPC1 has been characterized by the expression of several molecules that are associated with oligodendrocyte precursors, including Nestin and NG2. The cells are further characterized by their minimal or lack of expression of markers known to be present in other cell types, such as neurons, astrocytes, endoderm, mesoderm, and hESCs (Keirstead H S, Nistor G, Bernal G, Totoiu M, Cloutier F, Sharp K, Steward O. Human embryonic stem cell-derived oligodendrocyte progenitor cell transplants remyelinate and restore locomotion after spinal cord injury. J Neurosci. 2005 May 11; 25(19):4694-705; Zhang Y W, Denham J, Thies R S. Oligodendrocyte progenitor cells derived from human embryonic stem cells express neurotrophic factors. Stem Cells Dev. 2006 December; 15(6):943-52). In vitro, AST-OPC1 also produces diffusible factors that support neurite extension from sensory neurons (Zhang Y W, Denham J, Thies R S. Oligodendrocyte progenitor cells derived from human embryonic stem cells express neurotrophic factors. Stem Cells Dev. 2006 December; 15(6):943-52).

Pluripotent stem cell-derived neural cells have been used by researchers to treat CNS injuries and disorders in animal models. However, there remain obstacles in the development of such therapies for clinical applications in humans. To date, there are no commercially available therapies utilizing human pluripotent stem cell-derived differentiated cell populations for the treatment of spinal cord injury or other neurological conditions requiring CNS repair and/or remyelination.

### SUMMARY

In various embodiments described herein, the present disclosure provides, inter alia, a population of oligodendrocyte progenitor cells (OPCs) derived from pluripotent stem cells and methods of use of the same in the treatment of spinal cord injury.

In one embodiment, the present disclosure provides a method of improving upper extremity motor function in a human subject with a spinal cord injury, comprising administering to said subject a composition that comprises a population of allogeneic human oligodendrocyte progenitor cells (OPCs). In certain embodiments, the allogeneic human OPCs are capable of engrafting at a spinal cord injury site. In certain embodiments, administering the composition comprises injecting the composition into the spinal cord injury site. In some embodiments, the composition is injected approximately 2-10 mm caudal of the spinal cord injury epicenter. In further embodiments, the composition is injected approximately 5 mm caudal of the spinal cord injury epicenter. In some embodiments, the subject has a cervical spinal cord injury. In other embodiments, the subject has a thoracic spinal cord injury.

In certain embodiments, the composition is administered after the subject has suffered a traumatic spinal cord injury. In some embodiments, the composition is administered between 14-60 days after the spinal cord injury, such as between 14-30 days after the injury, such as between 20-40 days after the injury, such as between 40-60 days after injury. In certain embodiments, the composition is administered about 14, 15, 16, 17, 18, 19, 20, 21, 22, 23, 24, 25, 26, 27, 28, 29, 30, 31, 32, 33, 34, 35, 36, 37, 38, 39 or 40 days after the injury.

In certain embodiments, the improvement in the subject's upper extremity motor function may be measured as an increase or change over baseline in the subject's upper extremity motor score (UEMS) following the administration of the composition comprising allogeneic human OPCs. In some embodiments, the subject's UEMS detectably increases within 30-400 days from the administration of the composition. In some embodiments, the increase in the subject's UEMS is both detectable and significant over any potential increase in the UEMS of control subjects that were not administered a population of allogeneic human OPCs. In some embodiments, the subject's UEMS detectably increases within 30 days from the administration of the composition. In some embodiments, the subject's UEMS detectably increases within 60 days from the administration of the composition. In some embodiments, the subject's UEMS detectably increases within 90 days from the administration of the composition. In some embodiments, the subject's UEMS detectably increases within 180 days from the administration of the composition. In some embodiments, the subject's UEMS score detectably increases within 270 days from the administration of the composition. In some embodiments, the subject's UEMS score detectably increases within 360 days from the administration of the composition.

In certain embodiments, the subject's UEMS score continues to improve from the initial UEMS baseline measurement for a period of about 1-24 months post-administration of the composition comprising allogeneic human OPCs. In some embodiments, the subject's UEMS score improves over time following administration of allogeneic human OPC composition such that the baseline UEMS≤UEMS at 3 months≤UEMS at 6 months≤UEMS at 9 months≤UEMS at 12 months. In certain embodiments, the subject's UEMS score continues to improve up to or beyond 18 months post administration of the allogeneic human OPC composition. In certain embodiments, the subject's UEMS score continues to improve up to or beyond 24 months post administration of the allogeneic human OPC composition.

In certain embodiments, the subject's UEMS improvement over the course of 1-24 months post administration of allogeneic human OPCs can range from about 1 to about 30 points, such as about 2 points, such as about 4 points, such as about 6 points, such as about 8 points, such as about 10 points, such as about 12 points, such as about 14 points, such as about 16 points, such as about 18 points, such as about 20 points, such as about 22 points, such as about 24 points, such as about 26 points, such as about 28 points, such as about 30 points. In some embodiments, the subject's UEMS score improvement over the course of 1-18 months post administration of allogeneic human OPCs can be over 20 points.

In certain embodiments, the improvement in the subject's upper extremity motor function may be measured as improved motor level recovery (motor levels defined based on International Standards for Neurological Classification of Spinal Cord Injury (ISNCSCI). In some embodiments, the subject's motor level improvement is significant over any potential motor level improvement in control subjects that were not administered a population of allogenic human OPCs. In some embodiments, the subject's motor level improvement may be about one level at about 1-12 months post-administration of the allogeneic human OPC composition. In some embodiments, the subject's motor level improvement may be about two levels at about 1-12 months post-administration of the allogeneic human OPC composition. In some embodiments, the subject's motor level improvement may be more than two levels at about 1-12 months post-administration of the allogeneic human OPC composition. In some embodiments, the subject's measured motor level continues to improve from the initial baseline measurement for a period of about 1-24 months post-administration of the allogeneic human OPC composition, such as for about 1 month, for about 2 months, for about 3 months, for about 4 months, for about 5 months, for about 6 months, for about 7 months, for about 8 months, for about 9 months, for about 10 months, for about 11 months, for about 12 months, for about 13 months, for about 14 months, for about 15 months, for about 16 months, for about 17 months, for about 18 months, for about 19 months, for about 20 months, for about 21 months, for about 22 months, for about 23 months, or for about 24 months In some embodiments, the subject's measured motor level continues to improve from the initial baseline measurement for a period of about 12 months post-administration of the allogeneic human OPC composition. In some embodiments, the motor level improvement may be unilateral. In other embodiments, the motor level improvement may be bilateral.

In certain embodiments, the improvement in the subject's upper extremity motor function may be measured or assessed using means other than the UEMS or motor level recovery, including, but not limited to, various neurological exams and clinical impairment measurements such as GRASSP (the Graded Redefined Assessment of Strength, Sensibility and Prehension). In certain embodiments, the improvement in the subject's upper extremity motor function may be measured indirectly, such as by using MRI or by assessing the subject's functional independence using, for example, SCIM (spinal cord independence measure). Any means known in the art for detecting or assessing motor function improvement may be used.

In certain embodiments, the method further comprises administering to the subject a low dose immunosuppressant regimen. In certain embodiments, the immunosuppressant regimen comprises a dose of tacrolimus at about 0.03 mg/kg/day per os, adjusted to maintain a trough blood concentration of about 3-7 ng/mL through about day 46 following the administering of the composition, followed by tapering off and discontinuing the immunosuppressant at about day 60 following the administering of the composition comprising a population of allogeneically derived OPCs.

In certain embodiments, method comprises administering a composition comprising a population of allogeneic oligodendrocyte progenitor cells (OPCs), wherein the dose of the composition comprises between about 2×10⁶ and about 50×10⁶ AST-OPC1. In some embodiments, the dose of the composition comprises about 50×10⁶ AST-OPC1. In some embodiments, the dose of the composition comprises about 40×10⁶ AST-OPC1. In some embodiments, the dose of the composition comprises about 30×10⁶ AST-OPC1. In some embodiments, the dose of the composition comprises about 20×10⁶ AST-OPC1. In some embodiments, the dose of the composition comprises about 10×10⁶ AST-OPC1. In some embodiments, the dose of the composition comprises about 5×10⁶ AST-OPC1. In some embodiments, the dose of the composition comprises about 2×10⁶ AST-OPC1.

In certain embodiments, the OPCs are capable of remaining within the spinal cord injury site of said subject for a period of about 90 days or longer following the administration of the composition to the spinal cord injury site. In certain embodiments, the OPCs are capable of remaining within the spinal cord injury site of said subject for a period of about 1 year or longer following the administration of the composition to the spinal cord injury site. In further embodiments, the OPCs are capable of remaining within the spinal cord injury site of said subject for a period of about 2 years or longer following the administration of the composition to the spinal cord injury site. In further embodiments, the OPCs are capable of remaining within the spinal cord injury site of said subject for a period of about 3 years or longer following the administration of the composition to the spinal cord injury site. In further embodiments, the OPCs are capable of remaining within the spinal cord injury site of said subject for a period of about 4 years or longer. In yet further embodiments, the OPCs are capable of remaining within the spinal cord injury site of said subject for a period of about 5 years or longer.

In additional embodiments, the present disclosure provides a container comprising a composition comprising a population of allogeneic human oligodendrocyte progenitor cells (OPCs) that are capable of improving upper extremity motor function in a human subject with a spinal cord injury when administered to said subject. The OPCs of the present disclosure may be derived from any type of human pluripotent stem cell. In certain embodiments, the population of OPCs are the in vitro differentiated progeny of human embryonic stem cells (hESC). In other embodiments, the OPCs are the in vitro differentiated progeny of pluripotent stem cells other than human embryonic stem cells, such as induced pluripotent stem cells (iPSC). In certain embodiments, the subject has a cervical spinal cord injury. In other embodiments, the subject has a thoracic spinal cord injury.

### BRIEF DESCRIPTION OF DRAWINGS

For a fuller understanding of the nature and advantages of the present invention, reference should be had to the following detailed description taken in connection with the accompanying drawings.

FIG. 1 depicts a study design and timeline for a Phase 1/2a dose escalation study of AST-OPC1 in subjects with traumatic spinal cord injury.

FIG. 2 depicts study design with respect to subject cohorts and AST-OPC1 dosing. 2M=cohort subjects receive an injection of 2×10⁶ AST-OPC1; 10M=cohort subjects receive an injection of 10×10⁶ AST-OPC1; 20M=cohort subjects receive an injection of 20×10⁶ AST-OPC1. AIS A=American Spinal Injury Association (ASIA) Impairment Scale (AIS) grade A spinal cord injury, sensorimotor complete. AIS B=American Spinal Injury Association (ASIA) Impairment Scale (AIS) grade B spinal cord injury, motor complete, sensory incomplete. See, e.g. American Spinal Injury Association: International Standards for Neurological Classification of Spinal Cord Injury, revised 2000; Atlanta, Ga., Reprinted 2008.

FIG. 3 depicts AST-OPC1 injection procedure. Injections were performed using a table-mounted syringe-positioning device (SPD). Subjects in Cohorts 1 and 2 received a single intra-parenchymal injection into the spinal cord lesion, with an injection volume of 50 μl.

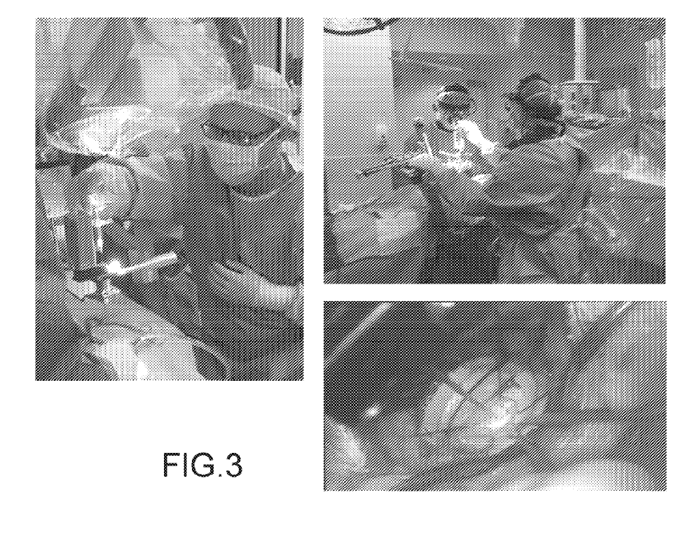

FIG. 4A and FIG. 4B show upper extremity motor function recovery data in Cohorts 1 and 2 as available in September 2016. FIG. 4A: All subjects in Cohort 1 (2×10⁶ AST-OPC1) and Cohort 2 (10×10⁶ AST-OPC1) exhibited improved upper extremity motor score (UEMS) relative to baseline. The average UEMS improvement at day 90 post AST-OPC1 injection equaled 5.0 points in Cohort 1 (N=3) and 9.5 points in Cohort 2 (N=4). FIG. 4 B: At 90 days post-injection, 50% (2 out of 4) of subjects in Cohort 2 had improved one motor level and 50% (2 out of 4) of subjects had improved two motor levels at least on one side. Motor levels were defined based on International Standards for Neurological Classification of Spinal Cord Injury (ISNCSCI; see Kirshblum, S C et al., International standards for neurological classification of spinal cord injury (Revised 2011). The Journal of Spinal Cord Medicine, 2011 34(6), 535-546). For UEMS and initial motor level/motor level improvement assessment, see Steeves J D et al., Extent of spontaneous motor recovery after traumatic cervical spinal cord injury, Spinal Cord 2011 49: 257-265; and Steeves J D et al., Outcome Measures for Acute/Subacute Cervical Sensorimotor Complete (AIS-A) Spinal Cord Injury During a Phase 2 Clinical Trial, Top Spinal Cord Inj Rehabil 2012; 18(1):1014.

FIG. 5 depicts matching criteria used to generate the closely matched historical controls from the EMSCI database.

FIG. 6A shows motor function recovery measured by a change in UEMS (+/−SEM) from baseline over time in Cohort 2 subjects compared to closely matched historical controls. Data are shown through 12 months of follow up. SEM=standard error of the mean.

FIG. 6B shows motor function recovery measured by a change in UEMS (+/−SEM) from baseline over time in Cohort 2 subjects compared to Cohort 1 subjects and closely matched historical controls. Data are shown through 12 months of follow up. SEM=standard error of the mean.

FIG. 7 shows motor function recovery measured as a percent of subjects improved by two or more motor levels in Cohort 2 subjects through 12-month follow-up visit. Cohort 2 subjects were compared to the closely matched historical controls from the EMSCI database.

### DETAILED DESCRIPTION

Before the present compositions and methods are described, it is to be understood that the present disclosure is not limited to the particular processes, compositions, or methodologies described, as these may vary. It is also to be understood that the terminology used in the description is for the purpose of describing the particular versions or embodiments only, and is not intended to limit the scope of the present invention which will be limited only by the appended claims. For example, features illustrated with respect to one embodiment may be incorporated into other embodiments, and features illustrated with respect to a particular embodiment may be deleted from that embodiment. Thus, the disclosure contemplates that in some embodiments of the disclosure, any feature or combination of features set forth herein can be excluded or omitted. In addition, numerous variations and additions to the various embodiments suggested herein will be apparent to those skilled in the art in light of the instant disclosure, which do not depart from the instant disclosure. In other instances, well-known structures, interfaces, and processes have not been shown in detail in order not to unnecessarily obscure the invention. It is intended that no part of this specification be construed to effect a disavowal of any part of the full scope of the invention. Hence, the following descriptions are intended to illustrate some particular aspects of the disclosure, and not to exhaustively specify all permutations, combinations and variations thereof.

Unless otherwise defined, all technical and scientific terms used herein have the same meaning as commonly understood by one of ordinary skill in the art to which this disclosure belongs. The terminology used in the description of the disclosure herein is for the purpose of describing particular embodiments only and is not intended to be limiting of the disclosure.

All publications, patent applications, patents and other references cited herein are incorporated by reference in their entireties.

Unless the context indicates otherwise, it is specifically intended that the various features of the disclosure described herein can be used in any combination. Moreover, the present disclosure also contemplates that in some embodiments of the disclosure, any feature or combination of features set forth herein can be excluded or omitted.

Methods disclosed herein can comprise one or more steps or actions for achieving the described method. The method steps and/or actions may be interchanged with one another without departing from the scope of the present invention. In other words, unless a specific order of steps or actions is required for proper operation of the embodiment, the order and/or use of specific steps and/or actions may be modified without departing from the scope of the present invention.

As used in the description of the disclosure and the appended claims, the singular forms “a,” “an” and “the” are intended to include the plural forms as well, unless the context clearly indicates otherwise.

As used herein, “and/or” refers to and encompasses any and all possible combinations of one or more of the associated listed items, as well as the lack of combinations when interpreted in the alternative (“or”).

The terms “about” and “approximately” as used herein when referring to a measurable value such as a percentages, density, volume and the like, is meant to encompass variations of ±20%, ±10%, ±5%, ±1%, ±0.5%, or even ±0.1% of the specified amount.

As used herein, phrases such as “between X and Y” and “between about X and Y” should be interpreted to include X and Y. As used herein, phrases such as “between about X and Y” mean “between about X and about Y” and phrases such as “from about X to Y” mean “from about X to about Y.”

The term “AST-OPC1” refers to a specific, characterized, in vitro differentiated cell population containing a mixture of oligodendrocyte progenitor cells (OPCs) and other characterized cell types obtained from undifferentiated human embryonic stem cells (uhESCs) according to specific differentiation protocols disclosed herein.

Compositional analysis of AST-OPC1 by immunocytochemistry (ICC), flow cytometry, and quantitative polymerase chain reaction (qPCR) demonstrates that the cell population is comprised primarily of neural lineage cells of the oligodendrocyte phenotype. Other neural lineage cells, namely astrocytes and neurons, are present at low frequencies. The only non-neural cells detected in the population are epithelial cells. Mesodermal, endodermal lineage cells and uhESCs are routinely below quantitation or detection of the assays.

The term “oligodendrocyte progenitor cells” (OPCs), as used herein, refers to cells of neuroectoderm/glial lineage that are committed to form progeny comprising mature oligodendrocytes. These cells typically express the characteristic markers NG2 and PDGF-Rα.

The term “therapeutically effective amount,” as used herein, refers to a dosage, dosage regimen, or amount sufficient to produce a desired result.

The terms “treatment,” “treat” “treated,” or “treating,” as used herein, can refer to both therapeutic treatment or prophylactic or preventative measures, wherein the object is to prevent or slow down (lessen) an undesired physiological condition, symptom, disorder or disease, or to obtain beneficial or desired clinical results. In some embodiments, the term may refer to both treating and preventing. For the purposes of this disclosure, beneficial or desired clinical results may include, but are not limited to one or more of the following: alleviation of symptoms; diminishment of the extent of the condition, disorder or disease; stabilization (i.e., not worsening) of the state of the condition, disorder or disease; delay in onset or slowing of the progression of the condition, disorder or disease; amelioration of the condition, disorder or disease state; and remission (whether partial or total), whether detectable or undetectable, or enhancement or improvement of the condition, disorder or disease. Treatment includes eliciting a clinically significant response. Treatment also includes prolonging survival as compared to expected survival if not receiving treatment.

The term “subject,” as used herein, refers to a human or an animal. In some embodiments, the term “subject,” refers to a male. In some embodiments, the term “subject,” refers to a female.

As used herein, “implantation” or “transplantation” refers to the administration of a cell population into a target tissue using a suitable delivery technique, (e.g., using an injection device).

As used herein, “engraftment” and “engrafting” refer to incorporation of implanted tissue or cells (i.e. “graft tissue” or “graft cells”) into the body of a subject. The presence of graft tissue or graft cells at or near the implantation site 180 days or later, post implantation, is indicative of engraftment. In certain embodiments, imaging techniques (such as, e.g. MRI imaging), can be used to detect the presence of graft tissue.

As used herein, “allogeneic” and “allogeneically derived” refer to cell populations derived from a source other than the subject and hence genetically non-identical to the subject. In certain embodiments, allogeneic cell populations are derived from cultured pluripotent stem cells. In certain embodiments, allogeneic cell populations are derived from hESCs. In other embodiments, allogeneic cell populations are derived from induced pluripotent stem (iPS) cells. In yet other embodiments, allogeneic cell populations are derived from primate pluripotent (pPS) cells.

The terms “central nervous system” and “CNS” as used interchangeably herein refer to the complex of nerve tissues that control one or more activities of the body, which include but are not limited to, the brain and the spinal cord in vertebrates.

Propagation and Culture of Undifferentiated Pluripotent Stem Cells

Methods of propagation and culture of undifferentiated pluripotent stem cells have been previously described. With respect to tissue and cell culture of pluripotent stem cells, the reader may wish to refer to any of numerous publications available in the art, e.g., Teratocarcinomas and Embryonic Stem cells: A Practical Approach (E. J. Robertson, Ed., IRL Press Ltd. 1987); Guide to Techniques in Mouse Development (P. M. Wasserman et al., Eds., Academic Press 1993); Embryonic Stem Cell Differentiation in Vitro (M. V. Wiles, Meth. Enzymol. 225:900, 1993); Properties and Uses of Embryonic Stem Cells: Prospects for Application to Human Biology and Gene Therapy (P. D. Rathjen et al., Reprod. Fertil. Dev. 10:31, 1998; and R. I. Freshney, Culture of Animal Cells, Wiley-Liss, New York, 2000).

In certain embodiments, a method can be carried out on a pluripotent stem cell line. In other embodiments, a method can be carried out on an embryonic stem cell line. In an embodiment, a method can be carried out on a plurality of undifferentiated stem cells that are derived from an H1, H7, H9, H13, or H14 cell line. In another embodiment, undifferentiated stem cells can be derived from an induced pluripotent stem cell (iPS) line. In another embodiment, a method can be carried out on a primate pluripotent stem (pPS) cell line. In yet another embodiment, undifferentiated stem cells can be derived from parthenotes, which are embryos stimulated to produce hESCs without fertilization.

In one embodiment, undifferentiated pluripotent stem cells can be maintained in an undifferentiated state without added feeder cells (see, e.g., (2004) Rosler et al., Dev. Dynam. 229:259). Feeder-free cultures are typically supported by a nutrient medium containing factors that promote proliferation of the cells without differentiation (see, e.g., U.S. Pat. No. 6,800,480). In one embodiment, conditioned media containing such factors can be used. Conditioned media can be obtained by culturing the media with cells secreting such factors. Suitable cells include, but are not limited to, irradiated (˜4,000 Rad) primary mouse embryonic fibroblasts, telomerized mouse fibroblasts, or fibroblast-like cells derived from pPS cells (U.S. Pat. No. 6,642,048). Medium can be conditioned by plating the feeders in a serum free medium, such as knock-out DMEM supplemented with 20% serum replacement and 4 ng/mL bFGF. Medium that has been conditioned for 1-2 days can be supplemented with further bFGF, and used to support pPS cell culture for 1-2 days (see. e.g., WO 01/51616; Xu et al., (2001) Nat. Biotechnol. 19:971).

Alternatively, fresh or non-conditioned medium can be used, which has been supplemented with added factors (such as, e.g., a fibroblast growth factor or forskolin) that promote proliferation of the cells in an undifferentiated form. Non-limiting examples include a base medium like X-VIVO™ 10 (Lonza, Walkersville, Md.) or QBSF™-60 (Quality Biological Inc. Gaithersburg, Md.), supplemented with bFGF at 40-80 ng/mL, and optionally containing SCF (15 ng/mL), or Flt3 ligand (75 ng/mL) (see, e.g., Xu et al., (2005) Stem Cells 23(3):315). These media formulations have the advantage of supporting cell growth at 2-3 times the rate in other systems (see, e.g., WO 03/020920). In one embodiment, undifferentiated pluripotent cells such as hESCs, can be cultured in a media comprising bFGF and TGFβ. Non-limiting example concentrations of bFGF include about 80 ng/ml. Non-limiting example concentrations of TGFβ include about 0.5 ng/ml.

In one embodiment, undifferentiated pluripotent cells can be cultured on a layer of feeder cells, typically fibroblasts derived from embryonic or fetal tissue (Thomson et al. (1998) Science 282:1145). Feeder cells can be derived, inter alia, from a human or a murine source. Human feeder cells can be isolated from various human tissues, or can be derived via differentiation of human embryonic stem cells into fibroblast cells (see, e.g., WO 01/51616). In one embodiment, human feeder cells that can be used include, but are not limited to, placental fibroblasts (see, e.g., Genbacev et al. (2005) Fertil. Steril. 83(5):1517), fallopian tube epithelial cells (see, e.g., Richards et al. (2002) Nat. Biotechnol., 20:933), foreskin fibroblasts (see, e.g., Amit et al. (2003) Biol. Reprod. 68:2150), and uterine endometrial cells (see, e.g., Lee et al. (2005) Biol. Reprod. 72(1):42).

Various solid surfaces can be used in the culturing of undifferentiated pluripotent cells. Those solid surfaces include, but are not limited to, standard commercially available cell culture plates, such as 6-well, 24-well, 96-well, or 144-well plates. Other solid surfaces include, but are not limited to, microcarriers and disks. Solid surfaces suitable for growing undifferentiated pluripotent cells can be made of a variety of substances including, but not limited to, glass or plastic such as polystyrene, polyvinylchloride, polycarbonate, polytetrafluorethylene, melinex, thermanox, or combinations thereof. In one embodiment, suitable surfaces can comprise one or more polymers, such as, e.g., one or more acrylates. In one embodiment, a solid surface can be three-dimensional in shape. Non-limiting examples of three-dimensional solid surfaces are described, e.g., in U.S. Patent Pub. No. 2005/0031598.

In one embodiment, undifferentiated stem cells can be grown under feeder-free conditions on a growth substrate. In one embodiment, a growth substrate can be Matrigel® (e.g., Matrigel® or Matrigel® GFR), recombinant Laminin, or Vitronectin. In another embodiment, undifferentiated stem cells can be subcultured using various methods such as using collagenase, or such as manual scraping. In another embodiment, undifferentiated stem cells can be subcultured using non-enzymatic means, such as 0.5 mM EDTA in PBS, or such as using ReLeSR™. In an embodiment, a plurality of undifferentiated stem cells are seeded or subcultured at a seeding density that allows the cells to reach confluence in about three to about ten days. In an embodiment, the seeding density can range from about 6.0×10³ cells/cm² to about 5.0×10⁵ cells/cm², such as about 1.0×10⁴ cells/cm², such as about 5.0×10⁴ cells/cm², such as about 1.0×10⁵ cells/cm², or such as about 3.0×10⁵ cells/cm² of growth surface. In another embodiment, the seeding density can range from about 6.0×10³ cells/cm² to about 1.0×10⁴ cells/cm² of growth surface, such as about 6.0×10³ cells/cm² to about 9.0×10³ cells/cm², such as about 7.0×10³ cells/cm² to about 1.0×10⁴ cells/cm², such as about 7.0×10³ cells/cm² to about 9.0×10³ cells/cm², or such as about 7.0×10³ cells/cm² to about 8.0×10³ cells/cm² of growth surface. In yet another embodiment the seeding density can range from about 1.0×10⁴ cells/cm² to about 1.0×10⁵ cells/cm² of growth surface, such as about 2.0×10⁴ cells/cm² to about 9.0×10⁴ cells/cm², such as about 3.0×10⁴ cells/cm² to about 8.0×10⁴ cells/cm², such as about 4.0×10⁴ cells/cm² to about 7.0×10⁴ cells/cm², or such as about 5.0×10⁴ cells/cm² to about 6.0×10⁴ cells/cm² of growth surface. In an embodiment, the seeding density can range from about 1.0×10⁵ cells/cm² to about 5.0×10⁵ cells/cm² of growth surface, such as about 1.0×10⁵ cells/cm² to about 4.5×10⁵ cells/cm², such as about 1.5×10⁵ cells/cm² to about 4.0×10⁵ cells/cm², such as about 2.0×10⁵ cells/cm² to about 3.5×10⁵ cells/cm², or such as about 2.5×10⁵ cells/cm² to about 3.0×10⁵ cells/cm² of growth surface.

Any of a variety of suitable cell culture and sub-culturing techniques can be used to culture cells in accordance with the present disclosure. For example, in one embodiment, a culture medium can be exchanged at a suitable time interval. In one embodiment, a culture medium can be completely exchanged daily, initiating about 2 days after sub-culturing of the cells. In another embodiment, when a culture reaches about 90% colony coverage, a surrogate flask can be sacrificed and enumerated using one or more suitable reagents, such as, e.g., Collagenase IV and 0.05% Trypsin-EDTA in series to achieve a single cell suspension for quantification. In an embodiment, a plurality undifferentiated stem cells can then be subcultured before seeding the cells on a suitable growth substrate (e.g., Matrigel® GFR) at a seeding density that allows the cells to reach confluence over a suitable period of time, such as, e.g., in about three to ten days. In one embodiment, undifferentiated stem cells can be subcultured using Collagenase IV and expanded on a recombinant laminin matrix. In one embodiment, undifferentiated stem cells can be subcultured using Collagenase IV and expanded on a Matrigel® matrix. In one embodiment, undifferentiated stem cells can be subcultured using ReLeSR™ and expanded on a Vitronectin matrix.

In one embodiment, the seeding density can range from about 6.0×10³ cells/cm² to about 5.0×10⁵ cells/cm², such as about 1.0×10⁴ cells/cm², such as about 5.0×10⁴ cells/cm², such as about 1.0×10⁵ cells/cm², or such as about 3.0×10⁵ cells/cm² of growth surface. In another embodiment, the seeding density can range from about 6.0×10³ cells/cm² to about 1.0×10⁴ cells/cm² of growth surface, such as about 6.0×10³ cells/cm² to about 9.0×10³ cells/cm², such as about 7.0×10³ cells/cm² to about 1.0×10⁴ cells/cm², such as about 7.0×10³ cells/cm² to about 9.0×10³ cells/cm², or such as about 7.0×10³ cells/cm² to about 8.0×10³ cells/cm² of growth surface. In yet another embodiment, the seeding density can range from about 1.0×10⁴ cells/cm² to about 1.0×10⁵ cells/cm² of growth surface, such as about 2.0×10⁴ cells/cm² to about 9.0×10⁴ cells/cm², such as about 3.0×10⁴ cells/cm² to about 8.0×10⁴ cells/cm², such as about 4.0×10⁴ cells/cm² to about 7.0×10⁴ cells/cm², or such as about 5.0×10⁴ cells/cm² to about 6.0×10⁴ cells/cm² of growth surface. In an embodiment, the seeding density can range from about 1.0×10⁵ cells/cm² to about 5.0×10⁵ cells/cm² of growth surface, such as about 1.0×10⁵ cells/cm² to about 4.5×10⁵ cells/cm², such as about 1.5×10⁵ cells/cm² to about 4.0×10⁵ cells/cm², such as about 2.0×10⁵ cells/cm² to about 3.5×10⁵ cells/cm², or such as about 2.5×10⁵ cells/cm² to about 3.0×10⁵ cells/cm² of growth surface.

### Oligodendrocyte Progenitor Cell Compositions

Methods to produce large numbers of highly pure, characterized oligodendrocyte progenitor cells from pluripotent stem cells have been previously described, for example, in U.S. patent application Ser. No. 15/156,316 and provisional patent application No. 62/315,454. Derivation of oligodendrocyte progenitor cells (OPCs) from pluripotent stem cells provides a renewable and scalable source of OPCs for a number of important therapeutic, research, development, and commercial purposes, including treatment of acute spinal cord injury.

In certain embodiments, the methods of producing highly pure populations of oligodendrocyte progenitor cells from pluripotent stem cells may comprise a pretreatment step during which the cells are incubated with one or more modulators of stem cell differentiation, for example, as described in U.S. provisional patent application No. 62/315,454, filed Mar. 30, 2016, and International patent application PCT/US2017/024986, filed Mar. 30, 2017.

In one embodiment, a cell population can have a common genetic background. In an embodiment, a cell population may be derived from one host. In an embodiment, a cell population can be derived from a pluripotent stem cell line. In another embodiment, a cell population can be derived from an embryonic stem cell line. In an embodiment, a cell population can be derived from a hESC line. In an embodiment, a hESC line can be an H1, H7, H9, H13, or H14 cell line. In another embodiment, a cell population can be derived from an induced pluripotent stem cell (iPS) line. In an embodiment a cell population can be derived from a subject in need thereof (e.g., a cell population can be derived from a subject that is in need to treatment). In yet another embodiment, a hESC line can be derived from parthenotes, which are embryos stimulated to produce hESCs without fertilization.

In certain embodiments, the OPCs of the present disclosure express one or more markers chosen from Nestin, NG2, Olig1 and PDGF-Rα. In certain embodiments, the OPCs of the present disclosure express all of the markers Nestin, NG2, Olig1 and PDGF-Rα. In some embodiments, at least 70% of AST-OPC1 are positive for Nestin expression. In some embodiments, at least 30% of AST-OPC1 are positive for NG2 expression. In some embodiments, at least 70% of AST-OPC1 are positive for Olig1 expression. In some embodiments, at least 70% of AST-OPC1 are positive for PDGF-Rα expression. The specific markers and combinations of various markers expressed by the cell populations of the present disclosure can be determined and quantified, for example, by flow cytometry.

Pharmaceutical Compositions

The OPCs can be administered to a subject in need of therapy per se. Alternatively, the cells of the present disclosure can be administered to the subject in need of therapy in a pharmaceutical composition mixed with a suitable carrier and/or using a delivery system.

As used herein, the term “pharmaceutical composition” refers to a preparation comprising a therapeutic agent or therapeutic agents in combination with other components, such as physiologically suitable carriers and excipients.

As used herein, the term “therapeutic agent” can refer to the cells of the present disclosure accountable for a biological effect in the subject. Depending on the embodiment of the disclosure, “therapeutic agent” can refer to the oligodendrocyte progenitor cells of the disclosure. Alternatively, “therapeutic agent” can refer to one or more factors secreted by the oligodendrocyte progenitor cells of the disclosure.

As used herein, the terms “carrier”, “pharmaceutically acceptable carrier” and “biologically acceptable carrier” may be used interchangeably and refer to a diluent or a carrier substance that does not cause significant adverse effects or irritation in the subject and does not abrogate the biological activity or effect of the therapeutic agent. In certain embodiments, a pharmaceutically acceptable carrier can comprise dimethyl sulfoxide (DMSO). In other embodiments, a pharmaceutically acceptable carrier does not comprise dimethyl sulfoxide. The term “excipient” refers to an inert substance added to a pharmaceutical composition to further facilitate administration of the therapeutic agent.

The therapeutic agent or agents of the present disclosure can be administered as a component of a hydrogel, such as those described in U.S. patent application Ser. No. 14/275,795, filed May 12, 2014, and U.S. Pat. Nos. 8,324,184 and 7,928,069.

The compositions in accordance with the present disclosure can be formulated for parenteral administration by injection, e.g., by bolus injection or continuous infusion. Formulations for injection can be presented in unit dosage form, e.g., in ampoules or in multi-dose containers, with an added preservative. The compositions can contain formulatory agents such as suspending, stabilizing and/or dispersing agents. In certain embodiments, the compositions can be formulated to be adapted for cryopreservation.

The compositions in accordance with the present disclosure can be formulated for administration via a direct injection to the spinal cord of a subject. In certain embodiments, a composition in accordance with the present disclosure can be formulated for intracerebral, intraventricular, intrathecal, intranasal, or intracisternal administration to a subject. In certain embodiments, a composition in accordance with the present disclosure can be formulated for administration via an injection directly into or immediately adjacent to an infarct cavity in the brain of a subject. In certain embodiments, a composition in accordance with the present disclosure can be formulated for administration through implantation. In certain embodiments, a composition in accordance with the present disclosure can be formulated as a solution.

In certain embodiments, a composition in accordance with the present disclosure can comprise from about 1×10⁶ to about 5×10⁸ cells per milliliter, such as about 1×10⁶ cells per milliliter, such as about 2×10⁶ cells per milliliter, such as about 3×10⁶ cells per milliliter, such as about 4×10⁶ cells per milliliter, such as about 5×10⁶ cells per milliliter, such as about 6×10⁶ cells per milliliter, such as about 7×10⁶ cells per milliliter, such as about 8×10⁶ cells per milliliter, such as about 9×10⁶ cells per milliliter, such as about 1×10⁷ cells per milliliter, such as about 2×10⁷ cells per milliliter, such as about 3×10⁷ cells per milliliter, such as about 4×10⁷ cells per milliliter, such as about 5×10⁷ cells per milliliter, such as about 6×10⁷ cells per milliliter, such as about 7×10⁷ cells per milliliter, such as about 8×10⁷ cells per milliliter, such as about 9×10⁷ cells per milliliter, such as about 1×10⁸ cells per milliliter, such as about 2×10⁸ cells per milliliter, such as about 3×10⁸ cells per milliliter, such as about 4×10⁸ cells per milliliter, or such as about 5×10⁸ cells per milliliter. In certain embodiments, a composition in accordance with the present disclosure can comprise from about 1×10⁸ to about 5×10⁸ cells per milliliter, such as about 1×10⁸ to about 4×10⁸ cells per milliliter, such as about 2×10⁸ to about 5×10⁸ cells per milliliter, such as about 1×10⁸ to about 3×10⁸ cells per milliliter, such as about 2×10⁸ to about 4×10⁸ cells per milliliter, or such as about 3×10⁸ to about 5×10⁸ cells per milliliter. In yet another embodiment, a composition in accordance with the present disclosure can comprise from about 1×10⁷ to about 1×10⁸ cells per milliliter, such as about 2×10⁷ to about 9×10⁷ cells per milliliter, such as about 3×10⁷ to about 8×10⁷ cells per milliliter, such as about 4×10⁷ to about 7×10⁷ cells per milliliter, or such as about 5×10⁷ to about 6×10⁷ cells per milliliter. In an embodiment, a composition in accordance with the present disclosure can comprise from about 1×10⁶ to about 1×10⁷ cells per milliliter, such as about 2×10⁶ to about 9×10⁶ cells per milliliter, such as about 3×10⁶ to about 8×10⁶ cells per milliliter, such as about 4×10⁶ to about 7×10⁶ cells per milliliter, or such as about 5×10⁶ to about 6×10⁶ cells per milliliter. In yet another embodiment, a composition in accordance with the present disclosure can comprise at least about 1×10⁶ cells per milliliter, such as at least about 2×10⁶ cells per milliliter, such as at least about 3×10⁶ cells per milliliter, such as at least about 4×10⁶ cells per milliliter, such as at least about 5×10⁶ cells per milliliter, such as at least about 6×10⁶ cells per milliliter, such as at least about 7×10⁶ cells per milliliter, such as at least about 8×10⁶ cells per milliliter, such as at least about 9×10⁶ cells per milliliter, such as at least about 1×10⁷ cells per milliliter, such as at least about 2×10⁷ cells per milliliter, such as at least about 3×10⁷ cells per milliliter, such as at least about 4×10⁷ cells per milliliter, or such as at least about 5×10⁷ cells per milliliter. In an embodiment, a composition in accordance with the present disclosure can comprise up to about 1×10⁸ cells or more, such as up to about 2×10⁸ cells per milliliter or more, such as up to about 3×10⁸ cells per milliliter or more, such as up to about 4×10⁸ cells per milliliter or more, such as up to about 5×10⁸ cells per milliliter or more, or such as up to about 6×10⁸ cells per milliliter.

In an embodiment, a composition in accordance with the present disclosure can comprise from about 4×10⁷ to about 2×10⁸ cells per milliliter.

In yet another embodiment, a composition in accordance with the present disclosure can have a volume ranging from about 10 microliters to about 5 milliliters, such as about 20 microliters, such as about 30 microliters, such as about 40 microliters, such as about 50 microliters, such as about 60 microliters, such as about 70 microliters, such as about 80 microliters, such as about 90 microliters, such as about 100 microliters, such as about 200 microliters, such as about 300 microliters, such as about 400 microliters, such as about 500 microliters, such as about 600 microliters, such as about 700 microliters, such as about 800 microliters, such as about 900 microliters, such as about 1 milliliter, such as about 1.5 milliliters, such as about 2 milliliters, such as about 2.5 milliliters, such as about 3 milliliters, such as about 3.5 milliliters, such as about 4 milliliters, or such as about 4.5 milliliters. In an embodiment, a composition in accordance with the present disclosure can have a volume ranging from about 10 microliters to about 100 microliters, such as about 20 microliters to about 90 microliters, such as about 30 microliters to about 80 microliters, such as about 40 microliters to about 70 microliters, or such as about 50 microliters to about 60 microliters. In another embodiment, a composition in accordance with the present disclosure can have a volume ranging from about 100 microliters to about 1 milliliter, such as about 200 microliters to about 900 microliters, such as about 300 microliters to about 800 microliters, such as about 400 microliters to about 700 microliters, or such as about 500 microliters to about 600 microliters. In yet another embodiment, a composition in accordance with the present disclosure can have a volume ranging from about 1 milliliter to about 5 milliliters, such as about 2 milliliter to about 5 milliliters, such as about 1 milliliter to about 4 milliliters, such as about 1 milliliter to about 3 milliliters, such as about 2 milliliter to about 4 milliliters, or such as about 3 milliliter to about 5 milliliters. In an embodiment, a composition in accordance with the present disclosure can have a volume of about 20 microliters to about 500 microliters. In another embodiment, a composition in accordance with the present disclosure can have a volume of about 50 microliters to about 100 microliters. In yet another embodiment, a composition in accordance with the present disclosure can have a volume of about 50 microliters to about 200 microliters. In another embodiment, a composition in accordance with the present disclosure can have a volume of about 20 microliters to about 400 microliters.

In certain embodiments, the present disclosure provides a container comprising a composition comprising a population of OPCs derived in accordance with one or more methods of the present disclosure. In certain embodiments, a container can be configured for cryopreservation. In certain embodiments, a container can be configured for administration to a subject in need thereof. In certain embodiments, a container can be a prefilled syringe.

For general principles in medicinal formulation, the reader is referred to Allogeneic Stem Cell Transplantation, Lazarus and Laughlin Eds. Springer Science+ Business Media LLC 2010; and Hematopoietic Stem Cell Therapy, E. D. Ball, J. Lister & P. Law, Churchill Livingstone, 2000. Choice of the cellular excipient and any accompanying elements of the composition will be adapted in accordance with the route and device used for administration.

In certain embodiments, the composition can also comprise or be accompanied by one or more other ingredients that facilitate the engraftment or functional mobilization of the enriched target cells. Suitable ingredients can include matrix proteins that support or promote adhesion of the target cell type or that promote vascularization of the implanted tissue.

### Uses of the Cells of the Present Disclosure

In various embodiments as described herein, the present disclosure provides methods of using a cell population that comprises pluripotent stem cell-derived OPCs for improving one or more neurological functions in a subject in need of therapy. In certain embodiments, methods for using pluripotent stem-cell derived OPCs in the treatment of traumatic spinal cord injury are provided. In other embodiments, methods for using pluripotent stem-cell derived OPCs in the treatment of other traumatic CNS injuries are provided. In other embodiments, methods for using pluripotent stem-cell derived OPCs in the treatment of non-traumatic CNS disorders or conditions are provided. In certain embodiments, a cell population in accordance with the present disclosure can be injected or implanted into a subject in need thereof.

In certain embodiments, methods for using pluripotent stem-cell derived OPCs in the treatment of conditions requiring myelin repair or remyelination are provided. The following are non-limiting examples of conditions, diseases and pathologies requiring myelin repair or remyelination: multiple sclerosis, the leukodystrophies, the Guillain-Barre Syndrome, the Charcot-Marie-Tooth neuropathy, Tay-Sachs disease, Niemann-Pick disease, Gaucher disease and Hurler syndrome. Other conditions that result in demyelination include but are not limited to inflammation, stroke, immune disorders, metabolic disorders and nutritional deficiencies (such as lack of vitamin B12). The OPCs of the present disclosure can also be used for myelin repair or remyelination in traumatic injuries resulting in loss of myelination, such as acute spinal cord injury.

The OPCs are administered in a manner that permits them to graft or migrate to the intended tissue site and reconstitute or regenerate the functionally deficient area. Administration of the cells can be achieved by any method known in the art. For example the cells can be administered surgically directly to the organ or tissue in need of a cellular transplant. Alternatively non-invasive procedures can be used to administer the cells to the subject. Non-limiting examples of non-invasive delivery methods include the use of syringes and/or catheters to deliver the cells into the organ or tissue in need of cellular therapy.

The subject receiving the OPCs of the present disclosure may be treated to reduce immune rejection of the transplanted cells. Methods contemplated include the administration of traditional immunosuppressive drugs such as, e.g., tacrolimus, cyclosporin A (Dunn et al., Drugs 61:1957, 2001), or inducing immunotolerance using a matched population of pluripotent stem cell-derived cells (WO 02/44343; U.S. Pat. No. 6,280,718; WO 03/050251). Alternatively a combination of anti-inflammatory (such as prednisone) and immunosuppressive drugs can be used. The OPCs of the invention can be supplied in the form of a pharmaceutical composition, comprising an isotonic excipient prepared under sufficiently sterile conditions for human administration.

Use in Treatment of CNS Traumatic Injury.

In certain embodiments, a cell population in accordance with the present disclosure can be capable of engrafting at a spinal cord injury site following implantation of a composition comprising the cell population into the spinal cord injury site.

In certain embodiments, a cell population in accordance with the present disclosure is capable of remaining within the spinal cord injury site of the subject for a period of about 90 days or longer following implantation of a dose of the composition into the spinal cord injury site. In other embodiments, a cell population in accordance with the present disclosure is capable of remaining within the spinal cord injury site of the subject for a period of about 1 year or longer following implantation of a dose of the composition into the spinal cord injury site. In further embodiments, a cell population in accordance with the present disclosure is capable of remaining within the spinal cord injury site of the subject for a period of about 2 years or longer following implantation of a dose of the composition into the spinal cord injury site. In further embodiments, a cell population in accordance with the present disclosure is capable of remaining within the spinal cord injury site of the subject for a period of about 3 years or longer following implantation of a dose of the composition into the spinal cord injury site. In further embodiments, a cell population in accordance with the present disclosure is capable of remaining within the spinal cord injury site of the subject for a period of about 4 years or longer following implantation of a dose of the composition into the spinal cord injury site. In yet further embodiments, a cell population in accordance with the present disclosure is capable of remaining within the spinal cord injury site of the subject for a period of about 5 years or longer following implantation of a dose of the composition into the spinal cord injury site.

In certain embodiments, a cell composition in accordance with the present disclosure is capable of improving upper extremity motor function in a human subject with a spinal cord injury when administered to said subject. In certain embodiments, the subject has a cervical spinal cord injury. In other embodiments, the subject has a thoracic spinal cord injury.

In one embodiment, the present disclosure provides a method of improving upper extremity motor function in a human subject with a spinal cord injury, comprising administering to said subject a composition that comprises a population of allogeneic human oligodendrocyte cells that are capable of engrafting at a spinal cord injury site. In certain embodiments, administering the composition comprises injecting the composition into the spinal cord injury site. In some embodiments, the composition is injected approximately 2-10 mm caudal of the spinal cord injury epicenter. In further embodiments, the composition is injected approximately 5 mm caudal of the spinal cord injury epicenter. In some embodiments, the subject has a cervical spinal cord injury. In other embodiments, the subject has a thoracic spinal cord injury.

In certain embodiments, the subject to whom a composition comprising a population of allogeneic human oligodendrocyte cells is administered to according to the methods of the present disclosure, gains an improvement in upper extremity motor function equal to at least one motor level (as defined based on International Standards for Neurological Classification of Spinal Cord Injury [ISNCSCI]). The improvement in function may be unilateral or bilateral. In other embodiments, the subject to whom a composition comprising a population of allogeneic human oligodendrocyte cells is administered to according to the methods of the present disclosure, gains an improvement in upper extremity motor function equal to at least two motor levels either unilaterally or bilaterally. In certain embodiments, the subject gains an improvement in upper extremity motor function equal to at least one motor level on one side and equal to at least two motor levels on the other side. In certain embodiments, the subject exhibits an improved upper extremity motor score (UEMS) relative to the subject baseline score prior to administration of a population of allogeneic human oligodendrocyte cells according to the methods of the present disclosure.

### Additional Embodiments

Additional embodiments of the present disclosure include the following:

1. A method of improving upper extremity motor function in a human subject with a traumatic spinal cord injury, the method comprising administering to said subject a therapeutically effective amount of a composition that comprises a population of allogeneic human oligodendrocyte progenitor cells.

2. The method according to 1, wherein administering the composition comprises injecting the composition into a spinal cord injury site.

3. The method according to 2, wherein the composition is injected approximately 2-10 mm caudal of the spinal cord injury epicenter.

4. The method according to 2, wherein the composition is injected approximately 5 mm caudal of the spinal cord injury epicenter.

5. The method according to any one of 1-4, wherein the human oligodendrocyte progenitor cells are capable of engrafting at the spinal cord injury site.

6. The method according to any one of 1-5, wherein the composition is administered between 15-60 days after the subject suffers a traumatic spinal cord injury.

7. The method according to 6, wherein the composition is administered between 20-40 days after the subject suffers a traumatic spinal cord injury.

8. The method according to any one of 1-7, further comprising administering to the subject a low dose immunosuppressant regimen.

9. The method according to any one of 1-8, wherein the composition comprises between about 2×10⁶ and 50×10⁶ AST-OPC1 cells.

10. The method according to 9, wherein the composition comprises about 10×10⁶ AST-OPC1 cells.

11. The method according to 9, wherein the composition comprises about 20×10⁶ AST-OPC1 cells.

12. The method according to any one of 1-11, wherein the subject has a cervical spinal cord injury.

13. The method according to any one of 1-12, wherein the subject's upper extremity motor function improves at least two motor levels by about 1-12 months after administering the composition.

14. The method according to 13, wherein the subject's motor level improvement is bilateral.

15. The method according to 13, wherein the subject's motor level improvement is unilateral.

16. The method according to 13, wherein the subject's upper extremity motor function improves by at least two motor levels by about 3 months after administering the composition.

17. The method according to 13, wherein the subject's upper extremity motor function improves by at least two motor levels by about 12 months after administering the composition.

18. The method according to any one of 1-17, wherein the allogeneic human oligodendrocyte progenitor cells are the in vitro differentiated progeny of pluripotent stem cells.

19. The method according to 18, wherein the pluripotent stem cells are human embryonic stem cells.

20. A pharmaceutical composition for use in improving upper extremity motor function in a human subject with a traumatic spinal cord injury, the composition comprising a population of allogeneic human oligodendrocyte progenitor cells.

21. The pharmaceutical composition according to 20, further comprising a biologically acceptable carrier.

22. The pharmaceutical composition according to 20-21, wherein the allogeneic human oligodendrocyte progenitor cells are the in vitro differentiated progeny of pluripotent stem cells.

23. The pharmaceutical composition according to 22, wherein the pluripotent stem cells are human embryonic stem cells.

24. A pharmaceutical composition for use in treatment of traumatic spinal cord injury in a human subject, the composition comprising a population of allogeneic human oligodendrocyte progenitor cells.

25. The pharmaceutical composition according to 24, further comprising a biologically acceptable carrier.

26. The pharmaceutical composition according to 24-25, wherein the allogeneic human oligodendrocyte progenitor cells are the in vitro differentiated progeny of pluripotent stem cells.

27. The pharmaceutical composition according to 26, wherein the pluripotent stem cells are human embryonic stem cells.

### EXAMPLES

The following examples are not intended to limit the scope of what the inventors regard as their invention nor are they intended to represent that the experiments below are all or the only experiments performed.

### Example 1: Phase 1/2A Escalation Dose Trial of AST-OPC1 in Patients with Motor Complete C4-C7 Cervical SCI

AST-OPC1 cells were generated by differentiation of WA01 (H1) hESCs from a master cell bank (MCB) as described in the U.S. patent application Ser. No. 15/136,316.

The initial clinical safety of AST-OPC1 was previously evaluated in a phase 1 clinical trial that enrolled patients with neurologically complete T3-T11 thoracic spinal cord injury (SCI). Based on favorable 5-year safety data from the phase 1 trial, a phase 1/2a trial was initiated to evaluate the safety and activity of escalating doses of AST-OPC1 in patients with sensorimotor complete C5-C7 cervical spinal injury.

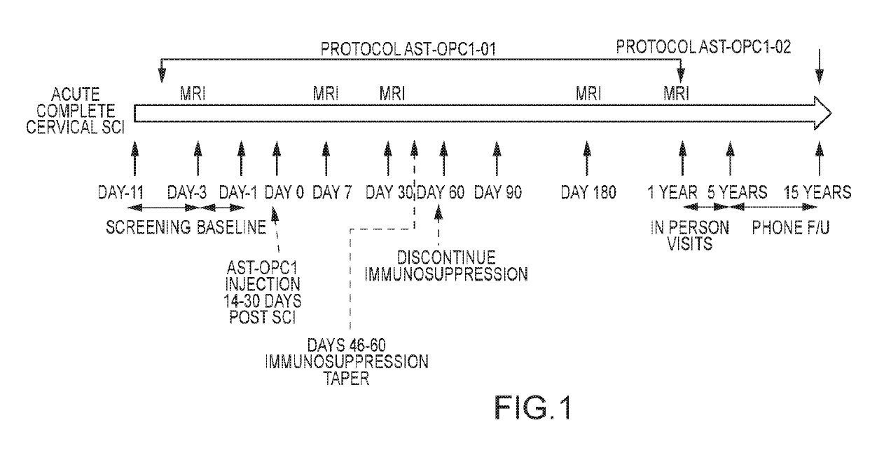

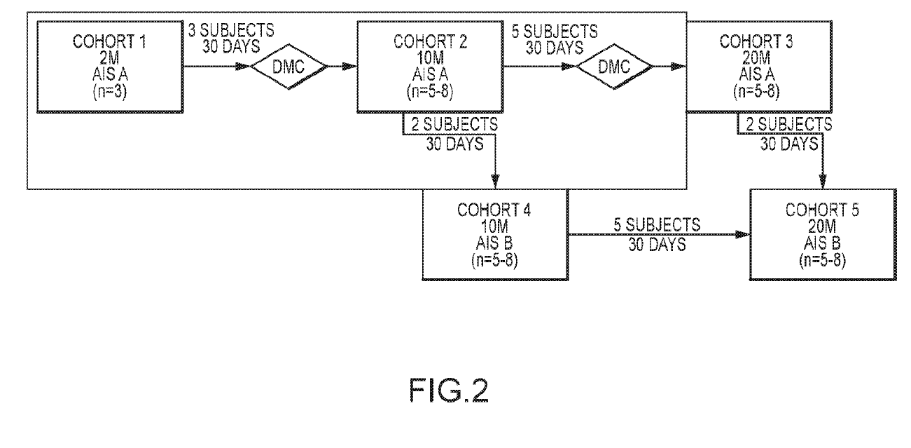

In the phase 1 trial, five subjects received a dose of 2×10⁶ AST-OPC1 between 7 and 14 days following their injury. The phase 1/2a trial has enrolled and will continue to enroll subjects to sequential dose cohorts receiving 2×10⁶, 10×10⁶ or 20×10⁶ AST-OPC1 between 14 and 40 days post-SCI. Study design of phase 1/2a trial is depicted in FIG. 1; cohort designs are depicted in FIG. 2. Subjects are followed for one year under the main study protocol and will be followed for an additional 14 years under a long term follow-up protocol.

In both phase 1 and phase 1/2a trials, AST-OPC1 has exhibited a strong safety profile.

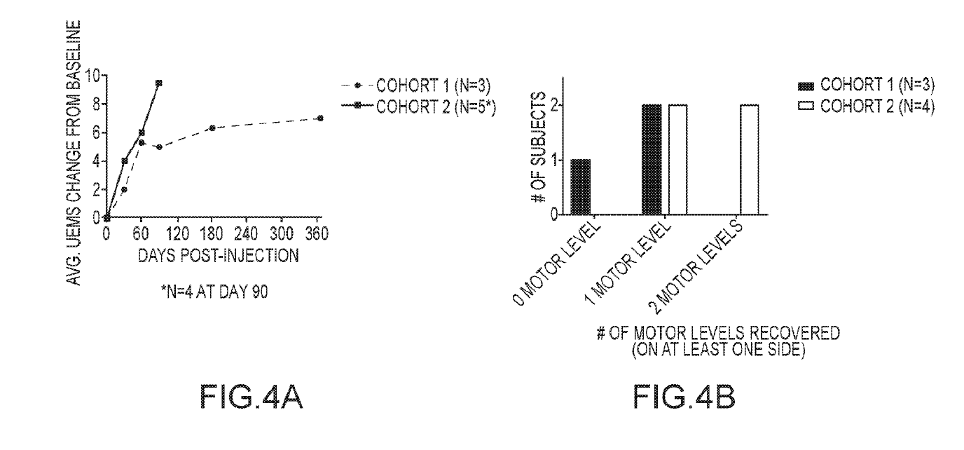

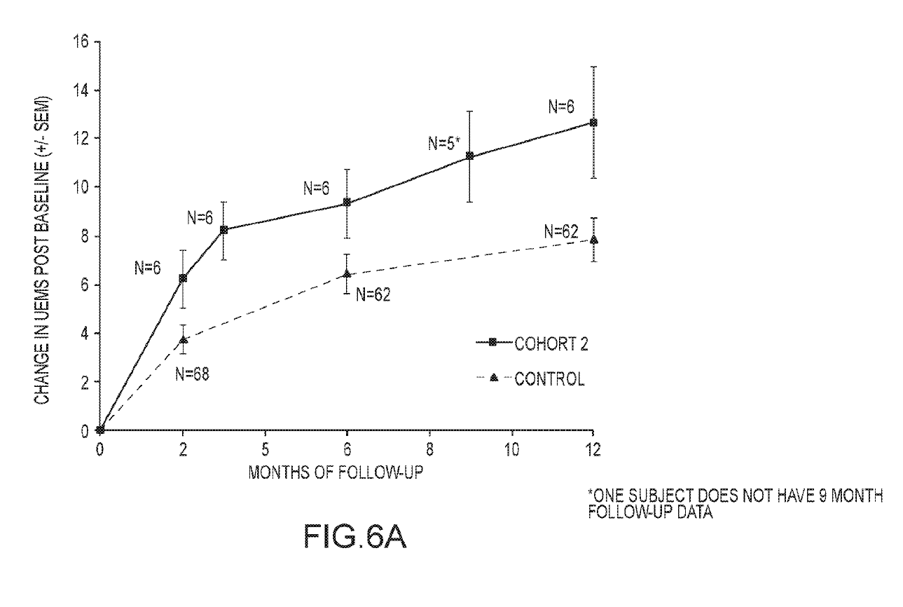

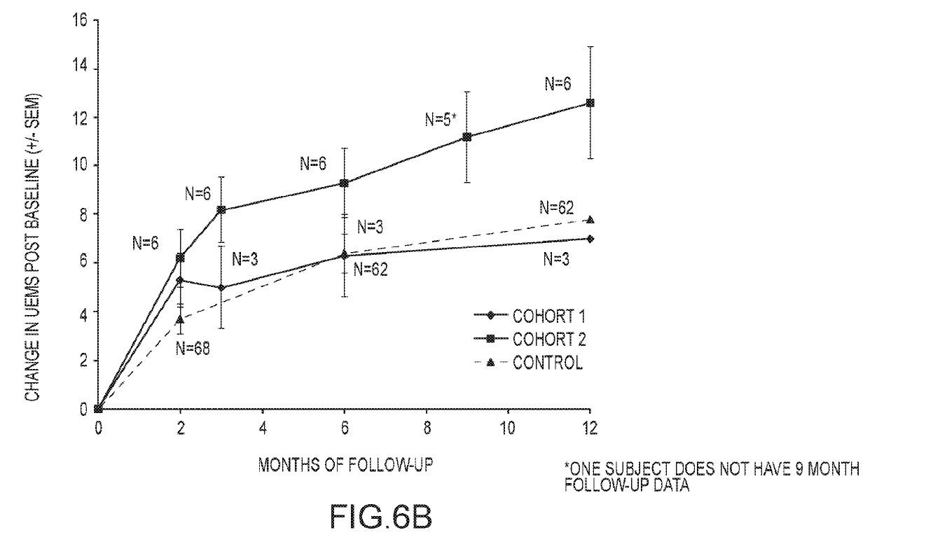

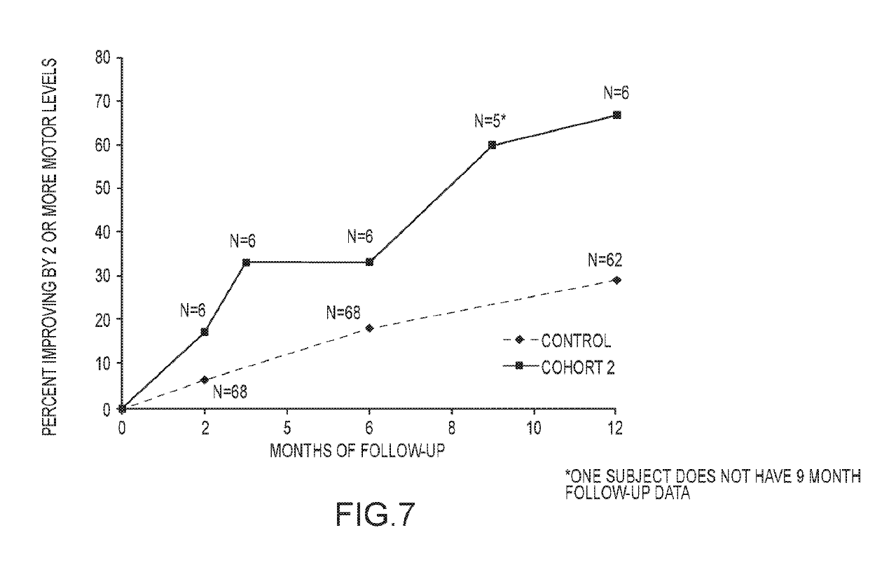

Early upper extremity motor function recovery results for Cohort 1 (2×10⁶ AST-OPC1) and Cohort 2 (10×10⁶ AST-OPC1) as available in September 2016 are presented in FIG. 4A (UEMS) and FIG. 4B (motor level recovery). Cohort 1 and 2 motor function recovery results through 12 months of follow up are presented in FIG. 6A and FIG. 6B (UEMS) and FIG. 7 (motor level recovery).

### Example 2: Comparison of Improvement in Upper Extremity Motor Function in Patients in Cohorts 1 and 2 Relative to Matched Historical Controls

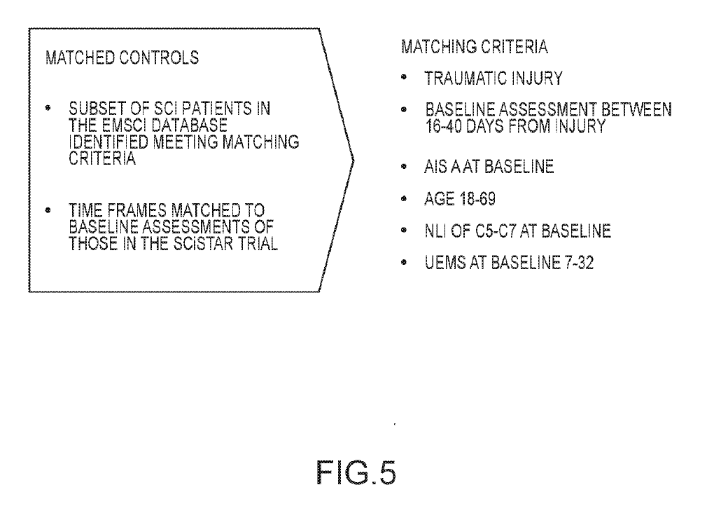

The motor function improvement in subjects in Cohorts 1 and 2 post-administration of AST-OPC1 was compared to a closely matched historical group of traumatic SCI patients derived from the EMSCI (European Multicenter Study about Spinal Cord Injury) database of over 3300 patients. The matching criteria used to generate the closely matched historical control data are shown in FIG. 5.

The comparison data through 12 months of follow up are presented in FIG. 6A, FIG. 6B and FIG. 7.

FIG. 6A: The motor function recovery in Cohort 2 subjects (10×10⁶ AST-OPC1) as measured by a change in UEMS over time compared favorably with the closely matched historical controls, with a significant improvement by 3 months, and continued increase through 12 months. FIG. 6B: As expected, the motor function recovery (UEMS) in Cohort 1 subjects (2×10⁶ AST-OPC1, same dose as used in the phase 1 safety trial) was similar to the matched historical controls, further supporting safety of AST-OPC1. Comparison of the improvement in motor scores between Cohorts 1 and 2 relative to the EMSCI historical control group supports an AST-OPC1 dose-dependent effect on the recovery of motor score. FIG. 7: The motor function recovery in Cohort 2 subjects was measured as an improvement in motor level over time vs. baseline measurement, through 12 months of follow-up. These improvements were compared to that of the closely matched historical controls from the EMSCI database. By 6 months after administration of AST-OPC1, 33% (2/6) of Cohort 2 subjects achieved a two or more level improvement in motor level on at least one side. By 12 months, 67% (4/6) of Cohort 2 subjects had achieved a two or more level improvement in motor level on at least one side. In comparison, 29% percent of the closely matched historical controls reached a two or more level improvement in motor level on at least one side by 12 months post SCI. The percentage of Cohort 2 subjects achieving two or more level improvement in motor function significantly exceeded both the motor level recovery in closely matched historical controls (29%) as well as recovery rates reported in the literature (26%, Steeves J D, et al. Outcome Measures for Acute/Subacute Cervical Sensorimotor Complete (AIS-A) Spinal Cord Injury During a Phase 2 Clinical Trial, Top Spinal Cord Inj Rehabil 2012; 18(1):1014).

1.-27. (canceled)

28. A method of improving upper extremity motor function in a human subject with a traumatic spinal cord injury, the method comprising administering to said subject a composition comprising a therapeutically effective amount of human oligodendrocyte progenitor cells derived from pluripotent stem cells.

29. The method of claim 28, wherein administering the composition comprises injecting the composition into a spinal cord injury site.

30. The method of claim 29, wherein the composition is injected approximately 2-10 mm caudal of the spinal cord injury epicenter.

31. The method of claim 30, wherein the composition is injected approximately 5 mm caudal of the spinal cord injury epicenter.

32. The method of claim 29, wherein the human oligodendrocyte progenitor cells are capable of engrafting at the spinal cord injury site.

33. The method of claim 28, wherein the composition is administered between 15-60 days after the subject suffers a traumatic spinal cord injury.

34. The method of claim 33, wherein the composition is administered between 20-40 days after the subject suffers a traumatic spinal cord injury.

35. The method of claim 28, further comprising administering to the subject a low dose immunosuppressant regimen.

36. The method of claim 28, wherein the composition comprises between about 2×10⁶ and 50×10⁶ cells.

37. The method of claim 36, wherein the composition comprises about 10×10⁶ cells.

38. The method of claim 36, wherein the composition comprises about 20×10⁶ cells.

39. The method of claim 28, wherein the subject has a cervical spinal cord injury.

40. The method of claim 28, wherein the subject's upper extremity motor function improves by at least two motor levels by about 1-12 months after administering the composition.

41. The method of claim 40, wherein the subject's motor level improvement is bilateral.

42. The method of claim 40, wherein the subject's motor level improvement is unilateral.

43. The method of claim 40, wherein the subject's upper extremity motor function improves by at least two motor levels by about 3 months after administering the composition.

44. The method of claim 40, wherein the subject's upper extremity motor function improves by at least two motor levels by about 12 months after administering the composition.

45. The method of claim 28, wherein the human oligodendrocyte progenitor cells are derived from human embryonic stem (hES) cells.

46. The method of claim 28, wherein the human oligodendrocyte progenitor cells are derived from induced pluripotent stem (iPS) cells.
